(19) United States
(12) Patent Application Publication
(10) Pub. No.: US 2021/0000579 A1
(43) Pub. Date: Jan. 7, 2021

(54) DENTAL ANESTHETIC DELIVERY DEVICE

(71) Applicant: Higdon Technologies LLC, Big Clifty, KY (US)

(72) Inventor: Norris Higdon, Big Clifty, KY (US)

(21) Appl. No.: 16/789,423

(22) Filed: Feb. 13, 2020

Related U.S. Application Data
(60) Provisional application No. 62/804,643, filed on Feb. 12, 2019.

(51) Int. Cl. A61C 19/08; A61M 5/30; A61M 37/00; A61M 19/00

(52) U.S. Cl. CPC A61C 19/08; A61M 5/3007; A61M 37/0015; A61M 19/00; A61M 2205/3653; A61M 2202/048; A61M 2205/366; A61M 2210/0631; A61M 2205/8206; A61M 2205/3606; A61M 2037/0023; A61M 2205/364

(57) ABSTRACT

Disclosed herein is a dental anesthetic delivery device. The dental anesthetic delivery device may comprise a handle and a body having a trough with a concave, horseshoe-shaped inner cavity. The inner cavity, which may be positioned over and around the area of a painful tooth, may further provide a soft or otherwise non-irritating interior surface which may comprise materials such as mesh, gauze, padding, or similar. The interior surface may act as a carrier of and delivery platform for various dental anesthetic substances. The dental anesthetic delivery device may incorporate a temperature modulation system that allows for the delivery of heat or cold to the affected area.

### RELATED APPLICATION

Under the provisions of 35 U.S.C. § 119(e), the Applicant claims the benefit of U.S. Provisional Patent Application No. 62/804,643, filed Feb. 12, 2019, which is incorporated herein by reference.

### FIELD OF DISCLOSURE

The present disclosure relates to a device for applying medicine to teeth or gums. More specifically, various embodiments of the present disclosure relate to a dental anesthetic delivery device. More specifically, the present disclosure relates to the field associated with the CPC classification A61 C19/063, medicament applicators for teeth or gums.

### BACKGROUND

Dental anesthetic delivery devices can be desirable for a number of reasons. Toothaches, ingrown wisdom teeth, inflamed gums, abscesses along the gum line, sore gums, dental cavities, and other painful dental ailments can cause extreme pain to an individual experiencing said symptoms. Utilizing dental anesthetic can mitigate those effects by reducing or temporarily numbing the pain. Further, because a dental care professional may not be immediately accessible, an individual so affected may benefit from over-the-counter (“OTC”) or otherwise self-applicable anesthetic modalities.

While OTC numbing gels such as benzocaine are presently available on the market to address providing short term relief for painful dental ailments, there are problems with traditional approaches to applying anesthetic directly to the affected area. Common problems include failure to maintain contact between the affected area and the analgesic substance, for sufficient duration and in sufficient concentration, to allow for effective pain relief. Additionally, other problems may include lack of ability to concomitantly apply heat or cold, which may work in tandem with a numbing agent to further ease swelling and relieve pain in the affected area.

A typical dental anesthetic applicator available to a person experiencing dental pain may be a cotton swab or similarly shaped tool used to apply the topical numbing anesthetic to the affected area, or simply the gel-dispensing exit point of a tube of analgesic. This modality fails to provide sufficient support for long term application of topical anesthetic. Further, in many currently available applicator configurations, they are limited to providing application to one affected tooth or gum at a time, the entire upper or lower pallet at once, or whatever locations in the oral cavity can be reached by maneuvering the tip of a gel-dispensing tube. Additionally, the potential relief from pain and swelling that may be afforded by heat or cold application is commonly obtained, if at all, from an ad hoc user solution like a warm washcloth, heating pad, or bag of frozen vegetables. Makeshift, difficult to use, or ineffective treatment solutions may encourage the introduction of non-sterile, sharp, breakable, dangerous, or otherwise unsuitable objects into an oral environment that may already be compromised.

Furthermore, there is a need to provide effective dental pain solutions for affected individuals who have limited access to emergency dental care. For example, those living in remote areas, rural communities, lower income areas, or areas with a shortage of dental care providers. Limited access to emergent dental care, coupled with a lack of proper tools to provide pain relief in a safe, effective application modality may cause a greater risk for infection or more severe dental problems.

A solution that improves the ability to apply medication to two or more teeth or gum areas at once, allows for long-term direct contact, long-term application, and optionally direct heating or cooling of the affected area, may therefore provide advantages over the traditional approaches.

In view of at least the above shortcomings, a need exists for a more effective dental anesthetic delivery device.

### BRIEF OVERVIEW

This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

Examples described herein include dental anesthetic delivery devices that address issues associated with the application of medicine to teeth and gums. Embodiments of the dental anesthetic delivery device disclosed herein may be supplied as stand-alone devices for applying dental anesthetic to teeth and gums. Alternatively, an embodiment of the dental anesthetic delivery device (or simply the “device”) may include a temperature modulating component. The temperature modulating component may be configured to fit within the body of the dental anesthetic delivery device. Additionally, the temperature modulating component may be configured such that it may be activated utilizing one or more triggering mechanisms.

One objective of the disclosed dental anesthetic delivery device may be to facilitate effective delivery of anesthetic to an area of a user's teeth and gums that is painful to the user (the “affected area”). Such effective delivery of anesthetic to an affected area may allow an individual experiencing dental discomfort (at times referred to herein as the “patient” or “user”), to keep the dental anesthetic delivery device in place for extended periods of time, including but not limited to minutes or hours.

Another objective of the disclosed dental anesthetic delivery device may be to provide for the delivery of one of, or a combination of medicament to the affected area.

Yet another objective of the disclosed dental anesthetic delivery device may be to provide sustained delivery of medicament by a substrate in substantially continuous contact with the affected area.

Still another objective of the disclosed dental anesthetic delivery device may be to provide release of medicament that is not constant over time, as on an increasing or decreasing curve, a proportionately larger initial release, or another time-variant delivery schedule.

Again another objective of the disclosed dental anesthetic delivery device may be to provide user-directed release of medicament, as by pressing a button or compressing the device between the jaws.

Further, an objective of the disclosed dental anesthetic delivery device may be to provide for the delivery of heat or cold to the affected area.

Further, another objective of the disclosed dental anesthetic delivery device may be to allow a user to treat teeth of the upper or lower jaw while comfortably resting the teeth of the opposite jaw on a surface of the dental anesthetic delivery device.

Further, yet another objective of the disclosed dental anesthetic delivery device may be to allow for a user to effectively apply medicament to two or more teeth or gums in an affected area in one instance.

Further, still another objective of the disclosed dental anesthetic delivery device may be to gently enclose the affected area within an inner cavity of the device.

Further, again another objective of the disclosed dental anesthetic delivery device may be to allow a user to apply pressure to the affected area by biting down on the dental anesthetic delivery device.

Even further, an objective of the disclosed dental anesthetic delivery device may be to allow for the expansion or narrowing of a medicament-bearing inner cavity, as by employing flexible or semirigid materials or by a user operated compression/release mechanism, so as to accommodate a variety of mouth sizes and the variation in cross-sectional width between front and rear teeth.

A dental anesthetic delivery device may comprise a handle, an applicator, and a delivery substrate. An applicator (at times referred to herein as the “head” of the device) may comprise a deeply concave, horseshoe-shaped inner cavity whose inner surface may comprise a delivery substrate. A delivery substrate may be placed in contact with and release medicament to an affected area. A delivery substrate may comprise material that is soft, gentle, or otherwise non-irritating to the affected area when placed in contact therewith, such as cotton padding, mesh, gauze, medical polymer, and the like.

A delivery substrate may comprise medicament for delivery to an affected area. In some embodiments, the substrate may be releasably loaded with pain-relieving medicament such as benzocaine, procaine, lidocaine, prilocaine, articaine, mepivacaine, eugenol, xylocaine, bupivacaine, marcain, bartinest, and the like.

In some embodiments, a dental anesthetic delivery device may further comprise an upper “accommodation” trough that can accommodate a comfortable resting position for the teeth of the jaw opposite the affected area (the “opposing teeth”, which belong to the “opposing jaw”).

In some embodiments, a user may manipulate the dental anesthetic delivery device by utilizing a handle to maneuver the dental anesthetic delivery device into position and apply the device to the affected area. A handle may allow the dental anesthetic delivery device to be angled in a way that allows for the dental anesthetic delivery device to be effectively positioned snugly around the teeth and gums of the affected area.

A dental anesthetic delivery device can incorporate a temperature modulation system (or “TMS”). In some instances, a TMS may comprise a heating modality. A TMS comprising a heating modality may contain a series of elements, including a power source, a transmission wire, and a heating element that allows for powering and delivery of heat to the affected area by the dental anesthetic delivery device. A TMS comprising a heating modality may contain chemical warming agents that provide heat via exothermic reactions, a hot fluid circulation system, or an applied-heat (e.g. microwavable) system, and so forth.

In some instances, a TMS may comprise a cooling modality. A TMS comprising a cooling modality may contain a chiller component, chemical cooling agents that extract heat via endothermic reactions, a gas expansion cooling component, a phase transition cooling component, a cold fluid circulation system, or an applied-cold (e.g. cooled in a freezer) system, and so forth.

A TMS comprising a heating or a cooling modality may contain agents that are able to retain and/or distribute hotter or colder temperatures (e.g. achieved via microwave, refrigeration of the device, hot water, ice, dry ice, etc.).

Additionally, in some instances a temperature modulation system may be configured such that it may be activated utilizing one or more triggering mechanisms including but not limited to a switch, button, or lever attached to the handle of the dental anesthetic delivery device. In some instances, a temperature modulating component may incorporate elements such as a photovoltaic cell, a fuel cell, or an inductive charging element.

In an embodiment, the dental anesthetic delivery device may incorporate a temperature modulation system in the form of a heat delivery mechanism. In further aspects, the heat delivery mechanism may be embedded in the cavity wall of the applicator of the dental anesthetic delivery device. In even further aspects, a triggering mechanism for the heat delivery mechanism may be embedded in the handle of the dental anesthetic delivery device.

Embodiments of the present disclosure provide assemblies that may comprise, but are not limited to, contoured fittings around mouth piece structures, features such as one or more handles comprising a swivel joint, soft and flexible handles, embedded rubber loops, and various other structures and components. Such an assembly may achieve various of the above-described objectives, or others.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the Applicants. The Applicants retain and reserve all rights in their trademarks and copyrights included herein, and grant permission to reproduce the material only in connection with reproduction of the granted patent and for no other purpose.

Furthermore, the drawings and their brief descriptions below may contain text or captions that may explain certain embodiments of the present disclosure. This text is included for illustrative, non-limiting, explanatory purposes of certain embodiments detailed in the present disclosure. In the drawings:

FIG. 1A illustrates a perspective view of an example dental anesthetic delivery device in use, in accordance with various embodiments of the present disclosure.

FIG. 1B illustrates a perspective view of an example dental anesthetic delivery device in use, in accordance with various embodiments of the present disclosure.

FIG. 2A illustrates a perspective view of an example dental anesthetic delivery device, in accordance with various embodiments of the present disclosure.

FIG. 2B illustrates a side elevation view of an example dental anesthetic delivery device, in accordance with various embodiments of the present disclosure.

FIG. 3A illustrates a cross-sectional side view of an example dental anesthetic delivery device in use, in accordance with various embodiments of the present disclosure.

FIG. 3B illustrates a cross-sectional side view of an example dental anesthetic delivery device, in accordance with various embodiments of the present disclosure.

FIG. 4 illustrates a perspective view of an example dental anesthetic delivery device in use, in accordance with various embodiments of the present disclosure.

FIG. 5A illustrates a top plan view of an example dental anesthetic delivery device, in accordance with various embodiments of the present disclosure.

FIG. 5B illustrates a top through view of an example dental anesthetic delivery device in use, in accordance with various embodiments of the present disclosure.

FIG. 5C illustrates a perspective view of an example dental anesthetic delivery device in use, in accordance with various embodiments of the present disclosure.

FIG. 5D illustrates a perspective view of an example dental anesthetic delivery device in use, in accordance with various embodiments of the present disclosure.

FIG. 5E illustrates an exploded view of a hinge assembly of an example dental anesthetic delivery device, in accordance with various embodiments of the present disclosure.

FIG. 5F illustrates an perspective view of a hinge assembly of an example dental anesthetic delivery device, in accordance with various embodiments of the present disclosure.

FIG. 6A illustrates an perspective through view of an example dental anesthetic delivery device having a temperature modulation system, in accordance with various embodiments of the present disclosure.

FIG. 6B illustrates an perspective through view of an applicator of an example dental anesthetic delivery device having a temperature modulation system, in accordance with various embodiments of the present disclosure.

FIG. 7 illustrates an perspective through view of an example dental anesthetic delivery device having a temperature modulation system, in accordance with various embodiments of the present disclosure.

FIG. 8 illustrates a flowchart depicting a method of using a dental anesthetic delivery device in accordance with various embodiments of the present disclosure.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of stages of various processes or methods that are described herein are illustrative and not restrictive. Accordingly, it should be understood that, although stages of various processes or methods may be shown and described as being in a sequence or temporal order, the stages of any such processes or methods are not limited to being carried out in any particular sequence or order, absent an indication otherwise. Indeed, the stages in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “stage for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of, embodiments of the present disclosure are not limited to use only in this context.

### I. Overview

### a. Dental Anesthetic Delivery Device

Consistent with embodiments of the present disclosure, a dental anesthetic delivery device 100 is provided. Various embodiments of device 100 are described herein. Components of the dental anesthetic delivery device 100 as presented in the following disclosure may be integrated, used independently, in conjunction with, used separately, or in connection with other embodiments they are not shown or described as functioning with. Any aspects of one embodiment may or may not be used interchangeably with other elements and aspects of a dental anesthetic delivery device 100 as presented in the present disclosure.

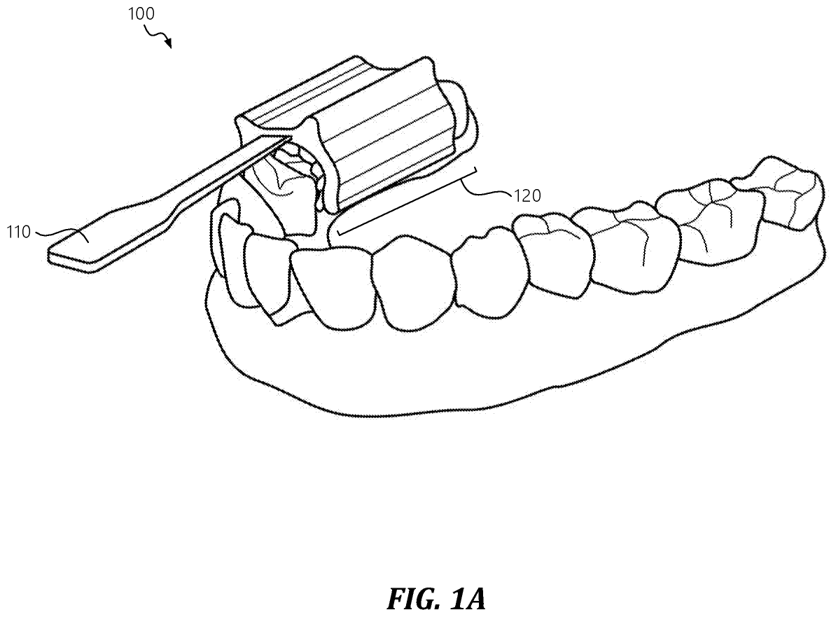

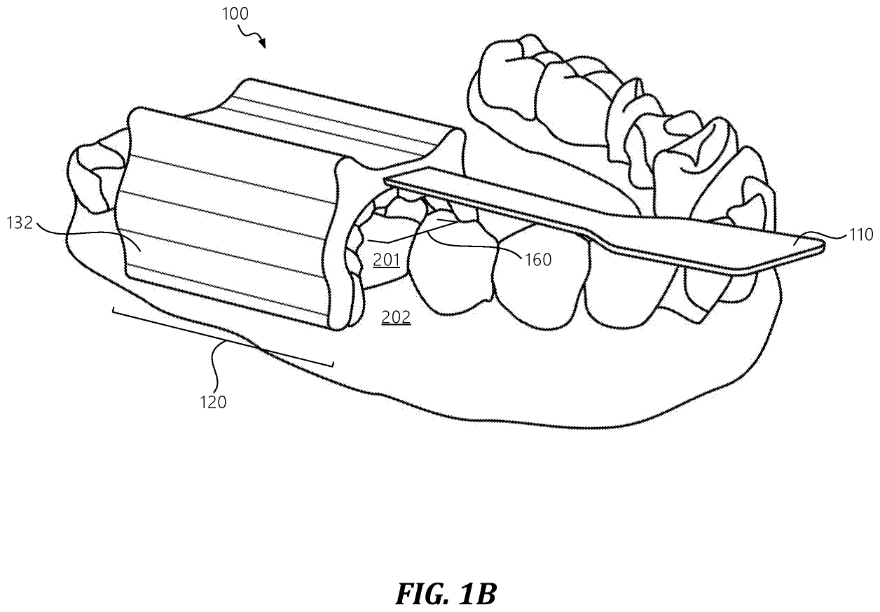

A dental anesthetic delivery device 100 may comprise in various embodiments. Device 100 may comprise components for applying medicament to areas of a user's teeth and gums. For example, device 100 may comprise an applicator 120 for delivery of medicaments to the affected area. In various embodiments, applicator 120 may comprise an inner cavity (or simply “cavity”) 160 which can accommodate the teeth and gums of the affected area, such that when so positioned, a portion of applicator 120 may contact an affected area (see, for example, FIG. 1B wherein device 100 contacts tooth 201 and gingiva 202).

An applicator 120 may comprise structural components, such as cavity wall 130, and a discrete or integral component that delivers medicament to an affected area of the user's teeth and gums, such as delivery substrate (or simply “substrate”) 140. Applicator 120 may comprise a temperature modulation system 180 that can apply heat or cold (at times referred to herein as “modulated temperature”) to the affected area.

Various embodiments of device 100 may be configured to deliver (e.g. via applicator 120) pain relieving or numbing agents to an affected area of a user's teeth and gums that is painful to the user. In an example consistent with embodiments depicted in FIG. 1, a user might use handle 110 to insert the applicator 120 of device 100 into their oral cavity, and to further maneuver applicator 120 so cavity 160 surrounds the gums and teeth of an affected area, bringing substrate 140 into contact with the affected area for delivery of its pain-relieving medicament payload (e.g. lidocaine or clove oil).

### b. Figure Overview

Referring now to FIGS. 1, 2, 3A-E, and 4, there are illustrated various embodiments of a dental anesthetic delivery device 100 and methods of use thereof.

Referring now to FIG. 1, there is illustrated in side elevation and perspective views 100a-c an embodiment of a dental anesthetic delivery device 100, depicting various structures that embodiments of device 100 may comprise—for example: a handle 110; an applicator 120; a cavity wall (or simply “wall”) 130 that may enclose an inner cavity 160, wherein the wall 130 may have an interior surface 131 and exterior surface 132; a delivery substrate 140 that may line interior surface 131; and an accommodation trough (or simply “trough”) 150 that may accommodate opposing teeth resting on trough surface 151 while device 100 is in use, wherein trough 150 may terminate at one or more sides with a trough lip 152.

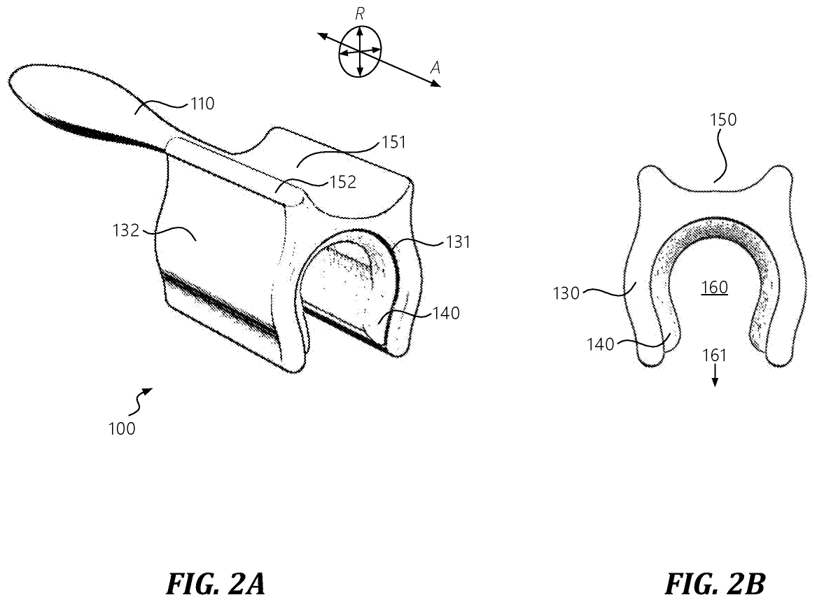

Referring now to FIG. 2, there are illustrated cross-sectional views 200a-d of various unseated and seated positions of an embodiment of dental anesthetic delivery device 100 in a user's mouth around the teeth 201 and gingiva 202. In various embodiments, applicator 120 may flex from an unseated or “closed” position as depicted in view 200b, to a seated or “open” position encompassing the teeth 201 and gingiva 202 of a user as depicted in views 200a, c, and d. Such flexion may allow cavity wall 130 to easily bend outward as the affected area is inserted into cavity 160, exerting gentle inward pressure sufficient to keep substrate 140 in contact with the affected area without causing additional discomfort. In some embodiments, a flexible, outwardly expansible cavity wall 130 having a more “closed” default state (as in view 200b) may allow for the accommodation of teeth and user mouths of various sizes.

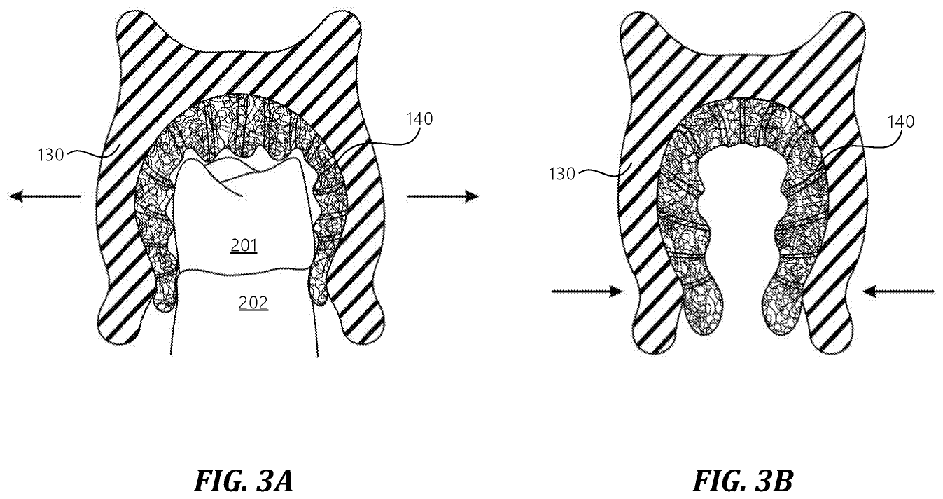

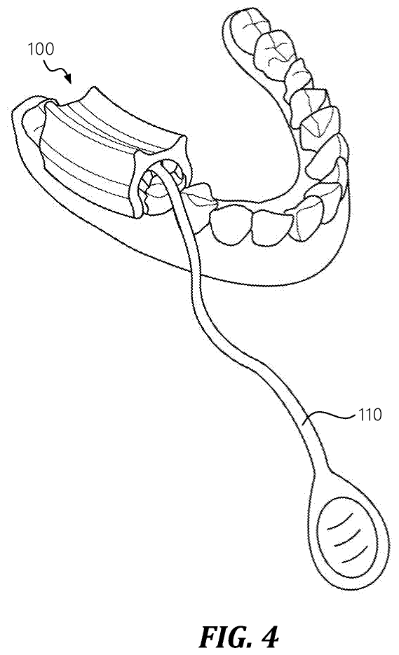

Referring now to FIG. 3A, there are illustrated views of four embodiments 301-304 of device 100, which variously illustrate features, systems, components, and configurations that may be present in various embodiments.

Referring now to FIG. 3B, there is illustrated in various views embodiment 301 of device 100, depicting various structures that embodiments of device 100 may comprise—for example: a temperature modulation system 180 capable of delivering heat to an affected area, wherein the TMS 180 may comprise power source 181, which may, upon engagement of triggering mechanism (which may be or comprise various mechanical, electrical, etc. user-operated switching or triggering modalities, but may be referred to herein simply as “button”) 184 by a user, transmit electrical power through transmission wire 182 to heating element (or simply “wire”) 183. As shown in embodiment 301, device 100 may comprise a maneuvering element such as hinge assembly (or simply “hinge”) 170. A hinge 170 may, by itself or in combination with other features, enable a user to more easily emplace applicator 120 in the oral cavity.

Referring now to FIG. 3C, there is illustrated in various views embodiment 302 of device 100, depicting various structures that embodiments of device 100 may comprise—for example: a hinge assembly 170, wherein the hinge assembly 170 may comprise structures such as a hinge pin (or simply “pin”) 171, a hinge cap (or simply “cap”) 172, and one or more through holes 173.

Referring now to FIG. 3D, there is illustrated in various views embodiment 303 of device 100, depicting various structures that embodiments of device 100 may comprise—for example: a handle 110 that is itself moldably flexible (i.e. able to be bent into, and substantially keep, a shape by a user). As in various embodiments, a TMS 180 may be present and may comprise a triggering mechanism such as button 184.

Referring now to FIG. 3E, there is illustrated in various views embodiment 304 of device 100, depicting various structures that embodiments of device 100 may comprise—for example: a TMS 180 that may comprise, i.a., a heating element 183 that may comprise wires embedded in wall 120. In some embodiments structurally consistent with embodiments illustrated in FIGS. 3A-E, TMS 180 may be configured to deliver cold in addition to, or instead of, heat.

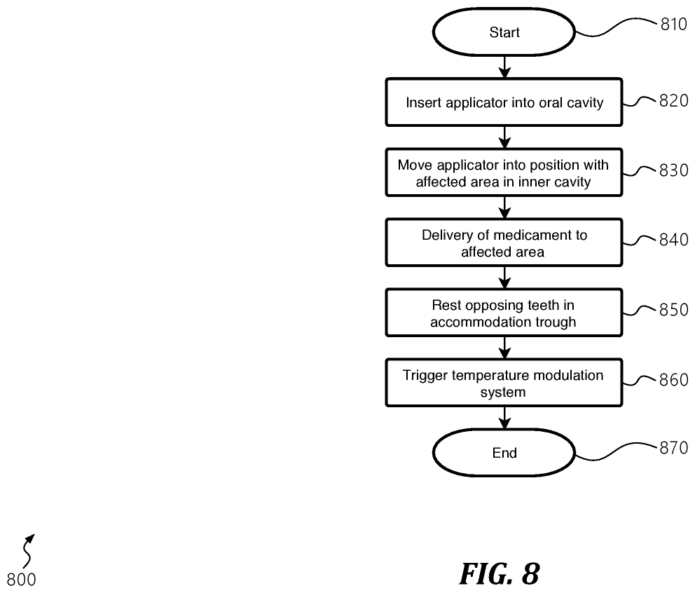

Referring now to FIG. 8, there are illustrated stages 810-870 of an illustrative method of using various embodiments of device 100.

### c. Material Composition

Device 100 and components thereof may comprise various materials and combinations thereof, including polymeric, metallic, ceramic, composite, wooden, and naturally occurring materials. Device 100 may include polymers of various description such as thermoplastic, thermoset, elastomer, natural, synthetic, rubberous, resinous, food-grade, medical-grade, non-toxic, and so on. In various embodiments, device 100 or components thereof (such as cavity wall 130) may be configured to have, or comprise materials that have, a Shore hardness ranging from 20A to 80A.

Various embodiments may comprise polymer materials, which may include silicone materials such as platinum catalyst silicone, cyclic olefin copolymer materials such as ethylene-norbornene copolymer, fluoropolymer materials such as PEEK and PTFE, biopolymer materials such as polylactic acid, and so forth. Polymer fabrication for device 100 may be achieved via a variety of methods, such as extrusion, compression molding, transfer molding, blow molding, injection molding, overmolding, transfer molding, thermoforming, sintering, additive manufacturing, foam expansion, and so forth.

Device 100 as a whole, or structures or components thereof, may comprise multiple materials. In one example, handle 110 may be constructed of a relatively stiffer polymer, while applicator 120 may be constructed of a relatively softer, more flexible polymer. In another example, cavity wall 130 may be constructed of a pliable polymer material but reinforced by metallic or stiffer polymer materials.

In embodiments that may be consistent with a device 100 comprising a temperature modulation system 180, portions of device 100 subject to modulated temperatures may comprise materials and configurations that can maintain, during and after operation of TMS 180, properties such as flexural, elastical, and dimensional stability sufficient to maintain gentle contact with the affected area, chemical and dielectrical stability, and low outgassing and leaching profiles so as to maintain the safe presence of device 100 in and around the oral cavity.

### d. Reusability

Device 100 may be disposable or reusable. In some embodiments which may be suitable for disposable use, some or all of the various components present in device 100 (which may include, e.g., handle 110, applicator 120, substrate 140, etc.) may be non-disassemblably joined, such that components so joined are not readily separated from each other. In examples consistent with various of such embodiments, handle 110 and the structure of applicator 120 may be of a unitary manufacture (as formed by e.g. a food-grade or medical silicone molding process).

In some embodiments that may be suitable for reusable use, some or all of the various components present in device 100 may be detachably joined, such that certain components can be (disposably, rechargably, or otherwise) replaced for use with certain other components. In an example consistent with various embodiments, a replaceable applicator 120 “head” may be detachably joined to a reusable handle 110 via a locking mechanism. In other consistent examples, replaceable components may comprise power source 181 (e.g. a battery), a chemical or fluid component of TMS 180, substrate 140 itself, or a medicamentous component of substrate 140.

Various configurations may be consistent with some disposable and some reusable embodiments. For example, substrate 140 may be fused, adhered, combined, or otherwise non-detachably coupled with interior surface 131—a configuration which may be consistent with both wholly inseparable (or “monolithic”) embodiments and with detachable embodiments having, e.g., a handle 110 that can detachably join to non-disassemblable “replacement” applicators 120.

### e. Shape, Appearance, and Other Appealing Characteristics

Dental anesthetic delivery device 100 in general, and handle 110 and applicator 120 in particular, may comprise elements that enhance the visual appearance or user appeal of device 100. In some embodiments, elements of device 100, such as handle 110 and the materials comprised thereby, may be configured to display (e.g. via a print process like dye diffusion or screen printing, a pyrographic process like wood burning, an electronic display technology like flexible OLED, etc.) graphics such as a cartoon character, a team logo, a patterned design, a company brand element, textual content, and so forth.

In some embodiments, elements of device 100 may have a physical shape intended to appeal to a user. For example, handle 110 may be shaped to resemble a magic wand or the grip of a baseball bat; applicator 120 may be shaped to resemble a character, an animal, a “fun” object like a rocketship, and so forth.

Some embodiments may comprise reflective, refractive, shimmering, sparkling, holographic, color-changing, iridescent, luminescent, or incandescent materials. Some embodiments may comprise compartments that display the visible flow of fluid therein. Some embodiments may have the capability of playing sound (e.g. via a speaker in handle 110, or via bone conduction through applicator 120).

Embodiments comprising features as described in this section may enhance or help facilitate user experience. For example, oral discomfort may deter a child from introducing an oral anesthetic delivery device 100 into the oral cavity, even though doing so may aid in relieving that discomfort. A device 100 featuring that child's favorite cartoon character, a playable rendition of a popular children's song, a transparent handle 110 encasing a small unicorn figure, or a light-up applicator 120 that can be seen through the cheek in the mirror may encourage a more positive and willing engagement with device 100. For another example, a user who is to regularly use device 100 (as with an embodiment comprising not pain-relieving but antimicrobial or antiseptic medicament) may enjoy one depicting indicia of their favorite sports team, character from a movie franchise, or hobby.

### f. Directional Terms

It may be useful to refer in some configurations to an “axial” direction, which may be envisioned as extending through applicator 120 from one end of cavity 160 to the other, along the direction indicated by the line labeled [symbol] in FIG. 2A). and a “radial” direction, extending in a plane perpendicular to the axial direction, along lines coplanar with those labeled [symbol] in FIG. 2A). The terminology as defined in this paragraph is for ease of explanation only and is not intended as limiting embodiments or configurations.

### II. Components

Some or all of the following components may be present in a dental anesthetic delivery device 100. The below description is in no way intended to limit the components that may be present in addition or in alternative to the listed components, nor to require that any particular component be included in a form described below or at all.

### a. Handle

A dental anesthetic delivery device 100 may comprise a handle 110. Handle 110 may comprise an elongated member and a base (whereupon, in some embodiments, an applicator 120 may be joined to handle 110). Handle 110 may be constructed from various materials and combinations thereof, including silicone. More specifically, handle 110 may comprise food-safe casting silicone of varying levels of hardness. For example, handle 110 may be configured to have Shore hardness ranging from 30A to 80A. In some embodiments, handle 110 may have a hardness in the high Shore A range and low Shore D range.

In some embodiments, handle 110 may comprise an elongated, substantially rigid protrusion for the user to hold and maneuver device 100 (see, for example, embodiments depicted in FIG. 1A and FIG. 7). A relatively more “simple” handle structure, as such, may provide advantages in cost of manufacture and user familiarity (i.e. suggest in an intuitive way how the device is intended to be used).

In various embodiments, handle 110 may allow for one or more degrees of freedom in positioning or repositioning applicator 120. In some embodiments, handle 110 may comprise an elongated protrusion that is flexibly moldable, which may allow a user to position, reposition, bend, or twist handle 110 (see, for example, embodiments depicted in FIG. 3D). Flexible moldability may be achieved in handles 110 comprising various configurations, such as polymer-encased metal wire, silicone putty, gooseneck structures, internal discrete state (or “click-in-place”) components, and so forth. In some embodiments, handle 110 may comprise a hinge assembly 170 that may allow for one or more degrees of freedom in position and/or rotation in each of one or more segments of handle 110 with respect to each other and/or applicator 120. Hinge 170 may comprise a wide variety of non-fixed couplings, such as knuckle joints, swivel joints, ball joints, turnbuckles, revolute joints, universal joints, and the like, and components such as pins, bearings, sliding or rotating elements, locking mechanisms, etc.

In some embodiments, device 100 may be configured such that oral insertion and maneuvering are accomplished by a handle 110 mechanism that is integral to applicator 120 (e.g. a thumb tab, finger grooves) rather than a discrete, extended handle 110.

That device 100 may be more easily manipulated and employed in the delivery of medicament and temperature modulation by way of handle 110 may be in keeping with the benefits of a dental anesthetic delivery device 100 as disclosed herein.

### b. Applicator, Cavity Wall, and Inner Cavity

An applicator 120 component of dental anesthetic delivery device 100 may comprise various shapes and materials (potentially multiple materials in a single embodiment). In some embodiments, applicator 120 may be constructed from silicone, soft plastic, rubber, foam, and the like. In one example, applicator 120 may comprise food- or medical-grade silicone. Cavity wall 130 may comprise material capable of conducting to the affected area modulated temperatures introduced by TMS 180.

Applicator 120 may comprise be embodied in, or comprise, various shapes and sections thereof, including (roughly) rectangular solid, cylindrical, ovoid, toroidal, irregular, or complex. Applicator 120 may comprise an inner cavity 160. Cavity 160 may form a deep concavity in the body of applicator 120, which may in some embodiments be substantially “horseshoe” shaped. Cavity 160 may be described as opening to the exterior of applicator 120 via cavity opening (or simply “opening”) 161, so as to accommodate ingress and egress of teeth and gums of the affected area into, out of, or through cavity 160.

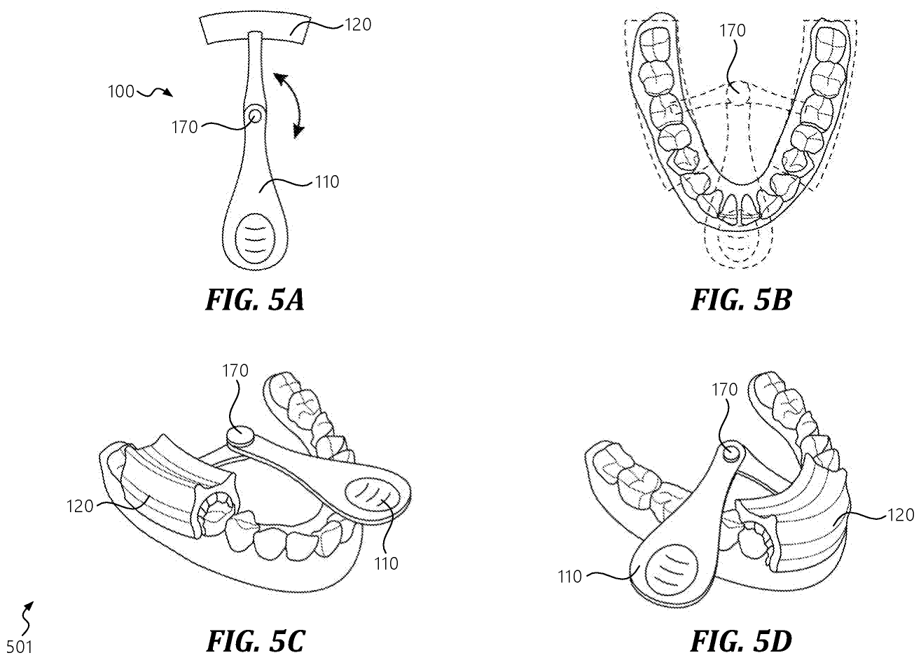

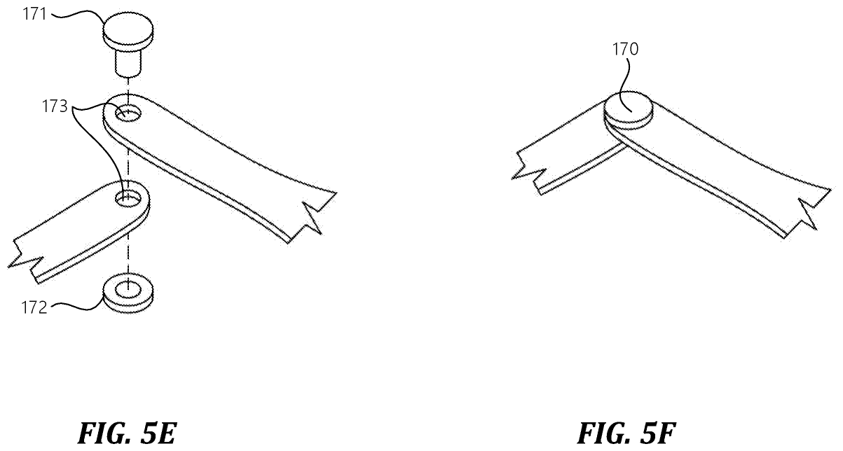

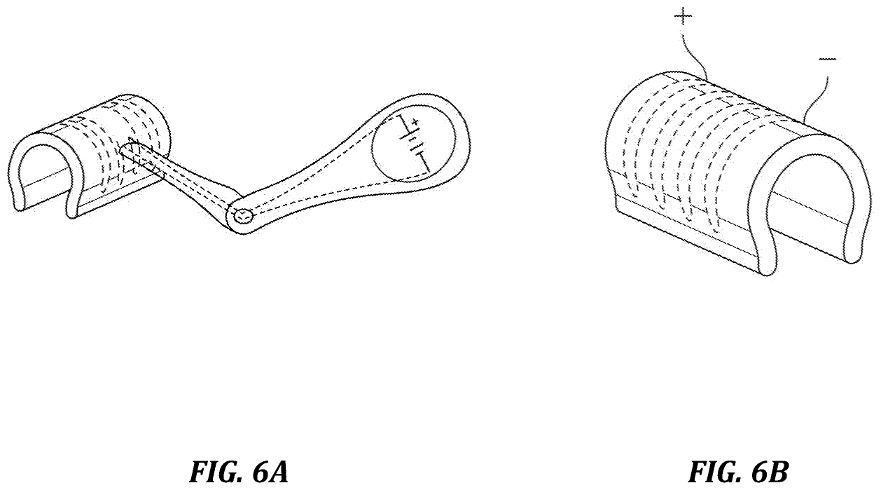

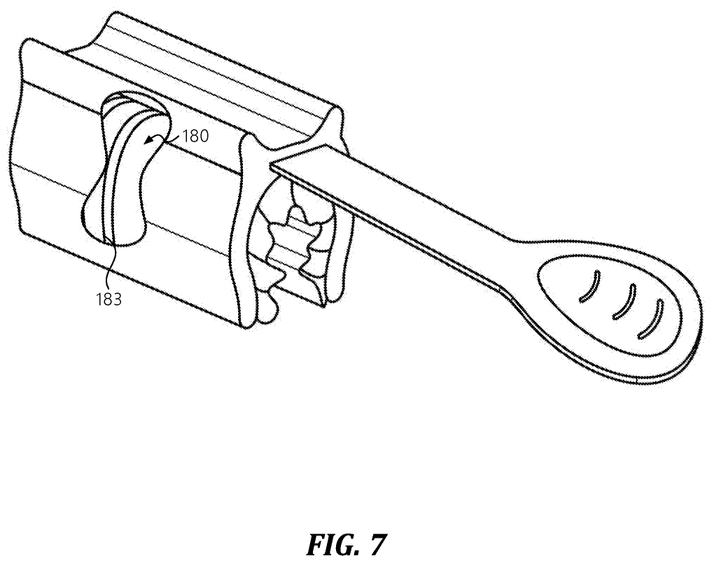

Consistent with various embodiments, and as visualized in FIG. 1, cavity 160 may open to the exterior of applicator 120 on three “sides”: at and through (along the axial direction) two “faces” of applicator 120, as well as the “bottom” of applicator 120. This type of opening configuration may also be visualized in FIG. 5A, where although applicator 120 is “axially curved,” the inner concavity may still be understood as opening axially at either end and at the bottom.

The interior void of cavity 160 may be bounded by cavity wall 130. In various embodiments, cavity 160 may be bounded by materials having qualities such as: rigidity sufficient to maintain the integrity of cavity 160 and contact with the affected area when in use; flexibility sufficient to allow emplacement of the applicator 120 without introducing significant compressive force to an (often very sensitive) affected area; and flexibility sufficient accommodate a variety of tooth size profiles (which may in some embodiments include accommodating tooth size profiles of children or young adults).

Various embodiments of device 100 may be configured to have applicators 120 (and possibly, commensurately, handles 110 and/or other features) of different sizes to accommodate different target users. For example, a device 100 intended for use by a small child may comprise an applicator 120 that is smaller than, and potentially made of different (e.g. softer) materials than, a device 100 intended for use by an adult.

Applicator 120 may comprise one or more sensors which may, e.g., provide feedback to the medicament release mechanism of substrate 140 or monitor temperature in connection with the operation of TMS 180. Some embodiments may comprise communicative connectivity such as Bluetooth or IR, which may enable the transmission of sensor information (e.g. for visualization) and user indications between device 100 and a computing device such as a smartphone or tablet.

In some embodiments, applicator 120 may be two sided, with opposing cavities 160 that comprise medicament-bearing and/or temperature modulating elements, to simultaneously accommodate upper and lower teeth.

Applicator 120 may comprise moving, rotating, vibrating, or retracting features. In some embodiments, gentle but sustained contact may be achieved via the mechanized excursion (during emplacement) and then incursion (when in place) of one or more walls 130. Embodiments featuring mechanized elements may comprise pressure or contact sensing capabilities to assist in bounding the motion of such elements.

In some embodiments, cavity 160 may be initially sealed, e.g. to maintain sterility or prevent escape of medicament substances. In an example, a user might peel a seal off of applicator 120 prior to insertion in the oral cavity. In another example, a under might insert applicator 120 and gently bite down in order that the teeth of the affected area might intrude through the seal into the cavity 160.

### c. Accommodation Trough

Dental anesthetic delivery device 100 may provide for an accommodation trough 150, which may comprise an open concavity on the upper surface of applicator 120. In various embodiments, trough 150 may be bounded by the body of applicator 120 (which may substantially be, or be contiguous with, cavity wall 130).

In some embodiments, a trough 150 may enable a patient to comfortably rest opposing teeth (those not being treated with medicament in cavity 160) on a surface of device 100. In some embodiments, the configuration of trough 150 and/or composition of cavity wall 130 may provide on applicator 120 with a non-hard surface into which a patient may bite down or apply pressure, which may in some instances provide the user some comfort.

### d. Substrate and Medicament

The interior surface 131 of applicator 120 may be lined with (or may itself comprise, in an integral fashion) substrate 140. Substrate 140 may comprise materials including but not limited to cotton padding, mesh, gauze, silk, medical- or food-grade polymers, woven materials, porous materials, fibrous materials, and so forth. In various embodiments, substrate 140 may be infused, releasably loaded, or combined with, or otherwise comprise, medicament for delivery upon contact with the affected area. Additionally or alternatively, some embodiments may permit or require a user to apply to or charge substrate 140 with, medicament.

In various embodiments, substrate 140 may be, or comprise structures that are, porous, fibrous, tubular, or wicking. In some embodiments, medicament may be drawn from a reservoir through a pathway (e.g. a tube or a wicking mechanism) to be delivered to the affected area. Substrate 140 may comprise elements for effectuating drug delivery as diffusors, jet injectors, microneedles or microneedle arrays (dissolving or otherwise), nanostructures, and so forth.

Medicament deliverable by device 100 (e.g. via a substrate 140 that is discrete or integral to applicator 120) may comprise various chemical and pharmaceutical preparations (prescription, over-the-counter, and otherwise), substances, structures, and materials (collectively, “agents”) including, without limitation: pharmaceutical agents; biologically active agents; excipients; adjuvants; stabilizers; carriers; binders; flavorants; drug delivery and transport facilitators; encapsulating, dissolving, and time-release agents; agents that react to introduction into an oral environment (including water- or saliva-activated agents); hydrophilic and hydrophobic agents; catalysts; anesthetic, numbing, pain-relieving, and otherwise analgesic agents; herbal and natural agents; anti-inflammatory agents; antiseptic agents; antibiotic agents; astringent agents; drying agents; lubricative agents; or other chemical agents. The term “medicament” is used interchangeably herein with the terms “dental anesthetic” and “dental anesthetic substances”.

In various embodiments, substrate 140 may comprise dental anesthetic substances such as benzocaine, procaine, lidocaine, prilocaine, articaine, mepivacaine, eugenol, phenol, xylocaine, bupivacaine, tetracaine, marcain, or bartinest.

Medicament may be released or imparted on various delivery profiles, including rapid release (i.e. a stronger initial release followed by a lower sustained dose), delayed release, multipeak, periodic, logarithmic, responsive (i.e. mediated by a sensor or feedback mechanism), and so forth. In an example, upon contact with the affected area, substrate 140 might release a quick, strong initial dose of dental anesthetic (which may be helpful in addressing with immediacy the pain that motivated the use of device 100) and a sustained delivery of dental anesthetic for a longer period of time (which may be helpful in continuing pain relief).

In another example consistent with some embodiments, a user may emplace applicator 120 in one location in the oral cavity for a short but sustained duration release of an antiseptic agent (e.g. triggered by a button) and then move applicator 120 to one or more additional locations and repeat. A device 100 consistent with the foregoing example may be configured for reuse by releasably storing numerous such “charges” of medicament.

Medicament may be distributed uniformly or heterogeneously with respect to the substrate 140. In some embodiments, medicament may be concentrated in particular areas such as near the bottom edge of cavity wall 130 (which region may be more likely to maintain continuing contact with gum tissue).

In various embodiments, device 100 may deliver a single type of medicament. In various other embodiments, device 100 may deliver multiple types of medicament; such multi-type delivery may involve simultaneous delivery of the different medicament types or on non-simultaneous release profiles for the different medicament types.

Some embodiments may include a system comprising a dial, release button, or other user-operated control that allows the user to influence the rate of release of medicament, or type of medicament released, to the affected area.

Medicament composition, delivery profile, and delivery method may vary between embodiments intended for use by different target users (e.g. children vs. adults). For example, an adult model of device 100 might be configured to deliver a relatively higher dose of benzocaine, while a pediatric model might be configured to deliver a dilute dose of clove oil or a relatively lower dose of benzocaine. In another example, while an adult model may be configured to deliver a pharmacological pain relieving agent, a pediatric model may be configured to deliver a non-pharmacological flavorant in conjunction with cooling from a TMS 180 (which may encourage a child to keep applicator 120 in the oral cavity when the cooling modality alone might not be sufficiently enticing).

### e. Temperature Modulation System

A dental anesthetic delivery device 100 may comprise a TMS 180 that encompasses heating and/or cooling modalities. In various embodiments, TMS 180 may be engaged by a user via a triggering mechanism 184 such as a mechanical, electrical, or electronic button or switch, a mechanical action such as twisting, rotating, squeezing, pulling, or biting down on a component, or by making an indication from a connected app or device (e.g. in an embodiment consistent with a device 100 comprising wifi, Bluetooth, or other communicative connectivity).

Some embodiments comprising a TMS 180 with a heating modality may include a resistive heating system comprising a power source 181, which may comprise features like a battery, a plug-in system, an ultracapacitor, a transformer, and various electrical and electronic components. In embodiments consistent with the foregoing, a heating element 183 such as a resistive wire embedded in cavity wall 130 and switchably connected to the terminals of a battery power source 181 may heat upon closing the circuit.

Some embodiments comprising a TMS 180 with a cooling modality may include an applied-cold system comprising a substance (e.g. a gel) internal to applicator 120 that may retain cold temperatures imparted to it in a refrigerator or freezer environment while substantially maintaining any overall flexibility and/or hardness characteristics of cavity wall 130 that may enable device 100 to be gently emplaced in an affected area.

A heating or cooling modality may be configured to deliver to the affected area or parts thereof temperatures that are, respectively, “safely above” (i.e. not above 44° C.), or “safely below” (i.e. not below 10° C.) the average oral temperature of approximately 37° C. TMS 180 may comprise a timer, a temperature feedback/limiter system, an analog or digital temperature indicator, various sensors, etc.

Some embodiments comprising a temperature modulation system 180 may comprise a vacuum chamber or gas outlet to accommodate reaction products or expanding gasses. Some embodiments comprising a gas expansion cooling system may include gas containment structures that are suitable for housing pressurized gas and impervious to being compromised by an errant user bite while inside the oral cavity. Some embodiments comprising an exothermic or endothermic chemical reaction system may comprise structures and materials configured to safely contain the chemical reactants and prevent their release even in the case of an errant user bite while inside the oral cavity.

### III. Methods of Use

As the dental anesthetic delivery device 100 may be applied to an affected area, a user may manipulate the device 100 by utilizing handle 110 to maneuver the device 100 into position. Handle 110 may allow the device 100 to be angled in a way that allows for effective positioning, for example, snugly around the teeth and gums of an affected area. With contact between the substrate 140 and the affected area, device 100 may deliver a sustained dose of medicament such as benzocaine that may provide for long lasting pain relief.

Referring now to FIG. 8, there is shown an exemplary method 800 of delivering medicament to an affected area via a dental anesthetic delivery device 100. Method 800 may begin at stage 810. At stage 820, a user may insert the end of device 100 comprising applicator 120 into their oral cavity.

At stage 830, the user may maneuver applicator 120 into position with respect to the affected area. For example, the user may position device 100 so that the teeth and gums of the affected area are within inner cavity 160. Cavity wall 130 may provide a snug but gentle fit around the affected area, allowing for contact between substrate 140 and the affected area.

At stage 840, substrate 140 may begin delivering medicament to the affected area. Substrate 140 may be impregnated with medicament substances (such as eugenol or lidocaine) that may be released to the affected area upon contact. Embodiments may release medicament to the affected area upon a variety of possible delivery profiles. In an example, dental medicament may be delivered on a rapid release profile in order to quickly address tooth or gum pain in the user's affected area.

At stage 850, the user may optionally, in embodiments whose elements so facilitate, rest (or potentially even bite down with) the teeth of the jaw opposite the affected area (which may then have, as per stage 830, teeth disposed in the cavity 160 of applicator 120). Embodiments may facilitate stage 850 by comprising an accommodation trough 150, although various embodiments may similarly permit comfortable resting of, or active compression involving, opposing teeth without comprising a trough 150. This may be the case where, for example, the upper area of applicator 120 comprises a particularly soft or cushioning material.

At stage 860, the user may optionally, in embodiments whose elements so facilitate, engage temperature modulation system 180 to deliver heat or cold to the affected area. In an example consistent with various embodiments, the user may trigger, by way of button 184 in handle 110 (or otherwise as described herein), the resistive heating of wire 183 embedded in cavity wall 130, providing heating to the affected area. In another example consistent with various embodiments, the user may trigger, by way of mechanical action (or otherwise as described herein), the endothermic cooling of chemical substances within device 100, providing cooling to the affected area.

Method 800 may conclude at stage 870. Notably, an end stage 870 of method 800 (which may comprise, for example, removing device 100 from the oral cavity) may occur a substantial time after the beginning stage 810, in some examples minutes or hours later. The duration and sustainability of positive effect (e.g. pain relief, reduction of inflammation, antimicrobial action, etc.) mediated by device 100 may be in keeping with the benefits of a dental anesthetic delivery device 100 as disclosed herein.

The order of stages presented are only illustrative of the possibilities and those steps can be executed or performed in any suitable fashion. Moreover, the various features of the examples described here are not mutually exclusive. Rather any feature of any example described here can be incorporated into any other suitable example. It is intended that the specification and examples be considered as exemplary only, with a true scope and spirit of the invention being indicated by the following claims.

### IV. Claims

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as example for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

The following is claimed:

1. A dental anesthetic delivery device comprising:
an applicator comprising:
a cavity wall;
an inner cavity bounded by the cavity wall; and
a substrate disposed on an interior surface of the cavity wall, wherein the substrate is configured to deliver medicament to an affected area in an oral cavity upon contact with the affected area; and
a handle coupled to the applicator.

2. The dental anesthetic delivery device of claim 1, wherein the applicator further comprises an accommodation trough dimensioned to accommodate opposing teeth during use of the dental anesthetic delivery device.

3. The dental anesthetic delivery device of claim 1, wherein the substrate comprises at least one of cotton padding, mesh, gauze, microfiber, and synthetic fabric.

4. The dental anesthetic delivery device of claim 1, wherein the substrate comprises at least one of a diffusor, a jet injector, and a microneedle.

5. The dental anesthetic delivery device of claim 1, wherein the cavity wall comprises at least one of polymeric, metallic, ceramic, composite, wooden, and naturally occurring materials.

6. The dental anesthetic delivery device of claim 5, wherein the cavity wall comprises polymeric material further comprising at least one of silicone, cyclic olefin copolymer, fluoropolymer, and biopolymer.

7. The dental anesthetic delivery device of claim 1, wherein the applicator comprises one or more materials having Shore A hardness ranging from 20A to 80A.

8. The dental anesthetic delivery device of claim 1, wherein the applicator is detachably coupled to the handle.

9. The dental anesthetic delivery device of claim 1, wherein the handle comprises a hinge assembly.

10. The dental anesthetic delivery device of claim 1, wherein the medicament comprises at least one of benzocaine, procaine, lidocaine, prilocaine, articaine, mepivacaine, eugenol, phenol, xylocaine, bupivacaine, tetracaine, marcain, and bartinest.

11. The dental anesthetic delivery device of claim 1, further comprising a temperature modulation system.

12. The dental anesthetic delivery device of claim 11, wherein temperature modulation system comprises a heating modality.

13. The dental anesthetic delivery device of claim 12, wherein the heating modality comprises at least one of a resistive heating system, an exothermic reaction heating system, a hot fluid circulation system, and an applied-heat system.

14. A dental anesthetic delivery system comprising:
a handle;
an applicator;
an exterior-opening inner cavity situated within the applicator;
a substrate disposed on an interior surface of the cavity; and
medicament.

15. The dental anesthetic delivery system of claim 14, further comprising a temperature modulation system.

16. The dental anesthetic delivery system of claim 14, further wherein the handle is flexibly moldable.

17. The dental anesthetic delivery system of claim 14, wherein the applicator comprises an accommodation trough.

18. A method for delivering medicament to an affected area of an oral cavity comprising:
inserting a dental anesthetic delivery device having an inner cavity comprising a medicament-bearing substrate into the oral cavity containing the affected area;
maneuvering the dental anesthetic delivery device so as to position at least a portion of the affected area within the inner cavity; and
delivering medicament to the affected area by bringing the at least a portion of the affected area into contact with the medicament-bearing substrate.

19. The method of claim 18, further comprising resting the opposing teeth in an accommodation trough of the dental anesthetic delivery device.

20. The method of claim 18, further comprising engaging a temperature modulation system of the dental anesthetic delivery device.
